(19) United States
(12) Patent Application Publication
(10) Pub. No.: US 2006/0292182 A1
(43) Pub. Date: Dec. 28, 2006

(54) Photoadjuvant immunotherapy

(76) Inventors: Lajos Kemeny, Szeged (HU); Zoltan Novak, Szeged (HU); Attila Dobozy, Szeged (HU); Zsolt Bor, Szeged (HU); Gavor Szabo, Szeged (HU); Bela Racz, Szeged (HU); Ferenc Ignacz, Melykut (HU)

Correspondence Address: Gergely T. Zimanyi;SIDLEY AUSTIN BROWN & WOOD LLP, Suite 2000, 555 California Street, San Francisco, CA, 94104-1715, US

(21) Appl. No.: 11/168,243

(22) Filed: Jun. 27, 2005

(51) Int. Cl. A61K 39/35

(52) U.S. Cl. 424/275.1; 607/86

(57) ABSTRACT

A photoadjuvant immunotherapeutical method includes the steps of providing a phototherapeutical apparatus, comprising a light source, an optical guidance system, and a patient interface; determining a minimal erythema dose; performing phototherapy by irradiating a target surface with the phototherapeutical apparatus; and performing immunotherapy by exposing the irradiated target surface to an antigen. The method can be applied to treat allergic diseases, including allergic rhinitis, rhinoconjunctivitis, asthma, and atopic dermatitis, as well as to treat autoimmune diseases. UVA, UVB and visible light can be used, emitted from a quartz bulb with a small discharge volume. The phototherapy increases the tolerance of a patient's body toward an antigen. During immunotherapy the irradiated target surface can be exposed to a naturally present antigen or to an administered antigen. The method can be used to treat RSA by preventing rejection of the fotus, or to suppress rejection reactions in organ or cell transplantation.

### BACKGROUND

1. Field of Invention

The present invention relates to phototherapeutical techniques to increase the efficacy of immunotherapies for the treatment of allergic and autoimmune disorders. The described method is also related to the prevention of immunologically mediated diseases of the body developing after cell and/or organ transplantations and also to the prevention of immune-mediated rejection of the embryo: spontaneous abortion.

2. Description of Related Art

The number of the patients with allergic diseases continues to increase, especially in the well-developed, industrialized countries. Although allergic diseases are not associated with severe morbidity and mortality, they have major effects on the quality of life. The increasing prevalence of allergic diseases, their impact on the quality of life and social costs underline the need for improved treatment options for these disorders. See A. W. Law, S. D. Reed, J. S. Sundy and K. A. Schulman: Direct costs of allergic rhinitis in the United States: estimates from the 1996 Medical Expenditure Panel Survey, J. Allergy Clin. Immunol., vol. 111, pp. 296-300 (2003); D. A. Stempel and R. Woolf: The cost of treating allergic rhinitis, Curr. Allergy Asthma Rep., vol. 2, pp. 223-230 (2002); A. Togias: Rhinitis and asthma: evidence for respiratory system integration, J. Allergy Clin. Immunol., vol. 111, pp. 1171-1183 (2003).

Allergic Diseases

Allergic diseases, including allergic rhinitis, rhinoconjunctivitis, asthma, atopic dermatitis and systhemic anaphylactic reactions, are among the most common health problems in many countries. Allergic diseases are high-cost, high-prevalence diseases affecting about 20-30% of the population. See A. B. Kay: Allergy and allergic diseases, N. Engl. J. Med., vol. 344, pp. 30-37 (2001).

The symptoms of some allergic diseases develop as follows. An allergen enters the body and induces the production of a specific Immunoglobulin E (IgE), which binds to specific receptors on the surface of mast cells. After subsequent exposure the allergen crosslinks the IgE receptors, resulting in the release of preformed mediators, such as histamine, from mast cells. These mediators are responsible for the development of the symptoms in patients. In addition, the mast cells may produce new inflammatory mediators as well, which attract further inflammatory cells into the mucous membrane. See P. H. Howarth, M. Salagean and D. Dokic: Allergic rhinitis: not purely a histamine-related disease, Allergy, vol. 55, pp. 7-16 (2000).

Allergic diseases are characterized by a type I, or immediate hypersensitivity, reaction that arises as a consequence of an allergen-IgE interaction in sensitized individuals. For the treatment of the disease, well-established symptomatic pharmacological therapies are available, using antihistamines, corticosteroids, decongestants and mast cell stabilizers. New therapeutic options have recently become increasingly important, including leukotriene modifiers, anti-IgE antibodies, phosphodiesterase inhibitors and intranasal heparin, and there have been developments in appropriate allergen-specific immunotherapy. See A. Schultz, B. A. Stuck, M. Feuring, K. Hormann and M. Wehling: Novel approaches in the treatment of allergic rhinitis, Curr. Opin. Allergy Clin. Immunol., vol. 3 , pp. 21-27 (2003).

For the treatment of allergic diseases many symptomatic treatments are available. Antihistamines are used locally or systemically for the blocking of the released mediators. For example, sodium cromoglycate is used to inhibit the release of mediators and corticosteroids are used locally or systemically for the blocking of the synthesis of new mediators. On the other hand, the presently known only curative treatment is the allergen specific immunotherapy (desensitizing therapy). At the same time, the presently available drugs often do not eliminate and cure the symptoms. Therefore, every new method for the treatment of this disease has considerable medical significance.

Autoimmune Diseases

Autoimmune diseases are characterized by immune responses to self antigens natively found in diseased individuals. Clinically significant autoimmune diseases include, for example, rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis or Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura. See e.g. W. E. Paul: Fundamental Immunology, Third Edition, Raven Press, New York, Chapter 30, pp. 1033-1097 (1993); and Cohen et al.: Autoimmune Disease Models, A Guidebook, Academic Press (1994).

The triggering factors of the autoimmune diseases are yet to be fully understood. It is supposed that autoimmune diseases might be triggered by infection, either clinical or sub-clinical. The infectious agent stimulates the immune system by presenting an epitope that is similar to an epitope natively present in some cell types in the individual. The stimulated immune system responds by destroying the self stuctures of the body, also called self proteins, glucoproteins, or “autoantigens,” resulting in a disease.

Some conventional therapies for autoimmune diseases focus on symptomatic relief or generalized immune suppression. Recently, new therapeutic approaches have been developed that are based on the premise that tolerance can be induced to the specific autoantigen, against which the immune system is acting.

Recurrent Spontaneous Abortion

Recurrent spontaneous abortion (RSA) is a common complication of pregnancy that may affect as many as 2% of women in their reproductive age. More than 50% of these cases seem to be mediated by enhanced immunity to the semialloantigen fetoplacental unit. This enhanced immunity results in an abnormal immune reaction agains the fetus, resulting in spontaneous abortions. During normal pregnancy in healthy individuals, maternal tolerance develops to the fetus by regulatory T cells. See V. R. Aluvihare, M. Kallikourdis, A. G. Betz: Regulatory T cells mediate maternal tolerance to the fetus, Nat. Immunol., vol. 3, pp. 266-71 (2004). In patients with RSA, this tolerance does not develop. In some of these cases this lack of tolerance may result in a spontaneous abortion.

Tolerance to the paternal antigens in RSA patients can be enhanced by paternal leukocyte immunization, which has been shown to significantly increase the live birth rate in RSA patients. However, the effectivity of the immunization needs to be increased. See M. K. Pandey, S. Agrawal: Induction of MLR-Bf and protection of fetal loss: a current double-blind randomized trial of paternal lymphocyte imunization for women with recurrent spontaneous abortion, Int. Immunopharmacol, vol. 4, pp. 289-298 (2004).

Immunotherapy

At present, many different forms of immunotherapy are used for the treatment of allergic and autoimmune diseases and for the treatment of spontaneous abortion. However, the efficacy of these treatments is low. Therefore, there is a need for new and more effective treatments.

In healthy individuals, the foreign antigens and self-antigens (autoantigens) activate negative regulatory functions that downregulate the immune response. Among these negative regulatory mechanisms, T regulatory lymphocytes (“Treg cells”) with suppressive functions play an important role. It has been shown that a reduced number of Treg cells is—at least partially—responsible for the development of allergic and autoimmune diseases. It also plays a role in the development of RSA. During specific immunotherapy for allergic and autoimmune diseases and for RSA, Treg cells are activated and have been suggested to be responsible for inducing tolerance toward these diseases.

In more detail, allergen specific immunotherapy (“SIT”) provides a curative treatment for allergic diseases. In SIT specific amounts of a known allergen are given in different time intervals to the allergic patients subcutaneously (specific subcutaneous immunotherapy=SCIT), in intranasal route (specific intranasal immunotherapy=SNIT) or sublingual route (sublingual immunotherapy=SLIT). These conventional immunotherapeutical methods have been found to be generally effective in the treatment of allergic rhinitis, allergic rhinoconjunctivitis, allergic chronic sinusitis, allergic asthma, atopic dermatitis, but also in the therapy of severe anaphylactic reactions. The mechanism of action of immunotherapies is believed to involve the generation of Treg cells with suppressive functions, increase in interleukin-10 (“IL-10”) production and increased synthesis of blocking immunoglobulin G (“IgG”) type antibodies.

In desensitization treatments, it is typically necessary for the patient to have frequent injections of the allergens in gradually increasing doses. For example, in some treatments initially there are injections every two or three days. Subsequently, the frequency is gradually reduced to once every two or three weeks. In the rush or ultrarush immuntherapeutical protocols the gradually increasing doses of allergen are given in a period of less than a few hours and result in quicker development of tolerance to a special allergen. See U.S. patent application Ser. No: US2003082212. Although the traditional subcutaneous route of allergen specific immunotherapy and the rush subcutaneous immunotherapy are burdened with the risk of severe adverse events, the SCIT therapy is still a frequently used and effective treatment for the therapy of different allergic diseases.

For the treatment of allergic diseases safer routes of allergen administration for immunotherapy have also been developed, which do not involve injections. For example, the specific nasal immunotherapy (SNIT) proved effective and safe in 17 of 18 controlled trials; thus it is considered a viable route of immunotherapy. See C. W. Canonica and G. Passalacqua: Noninjection routes for immunotherapy, J. Allergy Clin. Immunol., vol. 111, pp. 437-48 (2003).

The sublingual route of allergen specific immunotherapy (SLIT) is also supported by numerous controlled trials showing its efficacy in asthma and rhinitis in adults and children. The safety profile, assessed in clinical trials and postmarketing surveillance studies, is satisfactory. In a Cochrane study involving 22 clinical trials with 979 patients with allergic rhinitis, the SLIT therapy was found to be a safe and effective treatment. See D. R. Wilson, L. I. Torres, and S. R. Durham: Sublingual immunotherapy for allergic rhinitis, Cochrane Database Syst. Rev. vol. 2, p. CD002893 (2003). Sublingual immunotherapy is now accepted by the World Health Organization as a valid alternative to the subcutaneous route also in children. However, available allergen specific immunotherapies have disadvantages, including poor compliance, delayed and slight efficacy and patient frustration.

The immunotherapeutical methods used for the treatment of autoimmune diseases are based on the findings that mucosal administration of the autoantigens results in a tolerance induction to the given antigens by inducing regulatory T cells. For example, J. R. Haynes,, S. K. Prayaga, and I. A. Ramshaw describe effective methods for treating autoimmune diseasesin patent: WO9746253: Immunotherapy for Autoimmune Disease. This method is based on the induction of self antigen desensitization in patients by the introduction of the self antigen, or a gene coding thereof, into a cell of the patient. The antigen is selected on the basis of its involvement in the autoimmune process. The result of this immunotherapy includes tolerance induction that could result in a causative cure of the autoimmune disease. Unfortunately, tolerance induction by administration of self antigens or their antigenic epitopes can not be achieved in most patients, resulting in low or no clinical efficacy of these immunotherapies.

More than 50% of the recurrent spontaneous abortion (RSA) cases seem to be mediated by enhanced immunity to the semialloantigen fetoplacental unit. Tolerance to the paternal antigens can partly be achieved by paternal leukocyte immunization, which has been shown to significantly increase the live birth rate in RSA patients. See M. K. Pandey and S. Agrawal: Induction of MLR-Bf and protection of fetal loss: a current double-blind randomized trial of paternal lymphocyte imunization for women with recurrent spontaneous abortion, Int. Immunopharmacol. vol. 4, pp. 289-98 (2004). However, the method's effectivity needs to be increased.

Ultraviolet Light Induced Immunosuppression

Ultraviolet light has been used for more than twenty years for the treatment of allergic and auto-immune skin diseases. In various treatments and procedures ultraviolet-B light (280 nm-320 nm) and ultraviolet-A light (320 nm-400 nm) are used. The ultraviolet light inhibits the antigen-induced cellular immune response. See J. W. Streilein and P. R. Bergstresser: Genetic basis of ultraviolet-B on contact hypersensitivity, Immunogenetics, vol. 27, pp. 252-258 (1988).

A phototherapeutical apparatus and method for the treatment of inflammatory and hyperproliferative disorders of the body, such as allergic rhinitis and allergic rhinosinusitis, was described in U.S. patent application Ser. No. 10/410,690, by Lajos Kemény, Zsolt Bor, Gábor Szabó, Ferenc Ignácz, Béla Rácz, and Attila Dobozy, entitled: Phototherapeutical Apparatus and Method for the Treatment and Prevention of Diseases of Body Cavities, hereby incorporated by reference in its entirety; and in U.S. patent application Ser. No. 10/440,690, by Lajos Kemény, Zsolt Bor, Gábor Szabó, Ferenc Ignácz, Béla Rácz, and Attila Dobozy, entitled: Phototherapeutical Method and System for the Treatment of Inflammatory and Hyperproliferative Disorders of the Nasal Mucosa, also hereby incorporated by reference in its entirety (the “incorporated patents”). These incorporated patents show among others that ultraviolet light may inhibit the immediate type hypersensitivity reaction in IgE mediated allergic diseases.

The ultraviolet light suppresses the immune reaction by inhibiting the antigen presentation and by inducing T-cell apoptosis. Ultraviolet light was also shown to inhibit the contact hypersensitivity reaction in mice. See T. Schwarz: Utraviolet radiation-induced tolerance, Allergy, vol. 54, pp. 1252-61 (1999). In this delayed type hypersensitivity reaction the epicutaneous application of haptens to UV-exposed skin induced hapten-specific tolerance in mice. This was mediated via induction of regulatory T cells and via the inhibition correlated with the local expression of interleukin-10 (IL-10). It was also shown that ultraviolet radiation-induced regulatory T cells not only inhibit the induction but can also suppress the effector phase of contact hypersensitivity. See A. Schwarz, A. Maeda, M. K. Wild, K. Kernebeck, N. Gross, Y. Aragane, S. Beissert, D. Vestweber and T. Schwarz: Ultraviolet radiation-induced regulatory T cells not only inhibit the induction but can suppress the effector phase of contact hypersensitivity, J. Immunol. vol. 172, pp. 1036-43 (2004). The contact hypersentivity reaction is mediated by a type IV or a delayed type hypersensitivity reaction that differs from the mechanism of immediate type or type I hypersentitivity reactions. In spite of all these advances, the full potential of UV-based therapies has not been exhauseted yet.

### SUMMARY

Briefly and generally, embodiments of a photoadjuvant immunotherapeutical method include the steps of providing a phototherapeutical apparatus, comprising a light source, an optical guidance system, and a patient interface; determining a minimal erythema dose; performing phototherapy by irradiating a target surface with the phototherapeutical apparatus; and performing immunotherapy by exposing the irradiated target surface to an antigen.

The antigen can be one of an allergen, a modified allergen, a synthetic allergen, an autoantigen, a foreign antigen, and a modified antigen. The method can be applied to treat allergic diseases, such as allergic rhinitis, rhinoconjunctivitis, asthma, atopic dermatitis, and systhemic anaphylactic reactions. The method can also be applied to treat autoimmune diseases, including rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis, Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura.

The method can be practised with ultraviolet A, ultraviolet B and visible light, alone or in combination, emitted from a quartz bulb with electrodes, the electrodes defining a discharge volume in the range of about 0.1 mm³ to about 3 mm³.

The phototherapy includes irradiating a target surface of a patient with a dose, determined from the minimal erythema dose. The phototherapy increases the tolerance of a patient's body toward an antigen. During immunotherapy the irradiated target surface can be exposed to either a naturally present antigen or to an administered antigen. Some embodiments of the method are used to treat the nasal mucosa.

### BRIEF DESCRIPTION OF DRAWINGS

FIG. 1 illustrates steps of phototherapeutical method 100.

FIG. 2 illustrates an embodiment, where the irradiation of the oral mucosa of mice with UV and visible light induced an increase in the IL-10 mRNS production in the mucosa.

FIG. 3 illustrates an embodiment, where intranasal phototherapy using combined ultraviolet plus visible light for phototherapy for patients with allergic rhinitis results in an increase in the serum IL-10 level compared to placebo.

### DETAILED DESCRIPTION

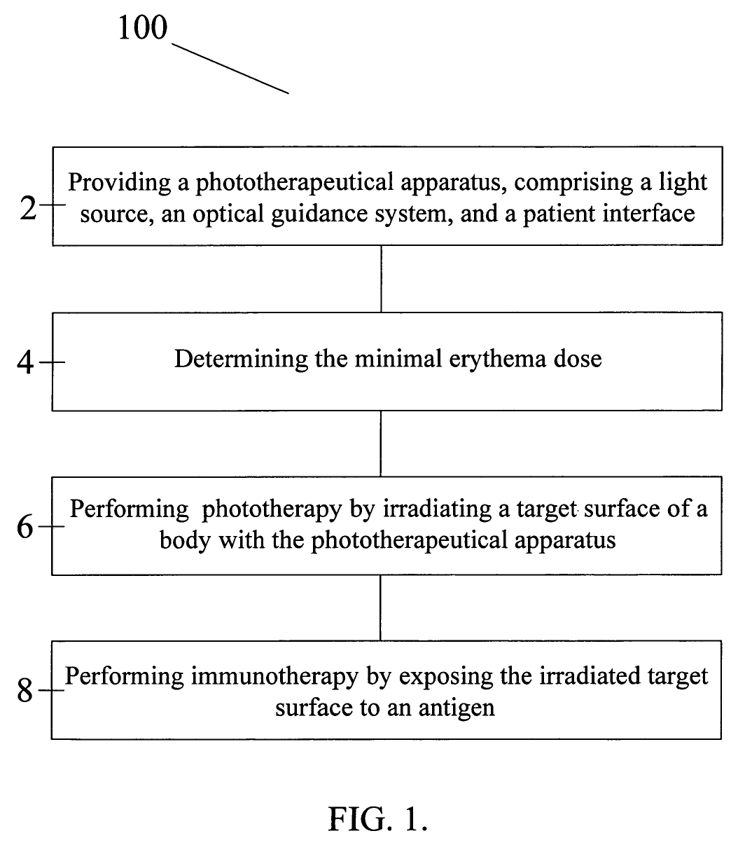

FIG. 1 illustrates a photoadjuvant immunotheraputical method 100 according to embodiments of the invention. The method includes a step 2 of providing a phototherapeutical apparatus, including a light source, an optical guidance system, and a patient interface, a step 4 of determining a minimal erythema dose, a step 6 of performing phototherapy by irradiating a target surface of a patient with the phototherapeutical apparatus, and a step 8 of performing immunotherapy by exposing the irradiated target surface to an antigen that might be an allergen, a modified allergen, an autoantigen or any other type of an antigen, for which the tolerance is to be induced.

In embodiments, method 100 can be applied to treat allergic diseases, including: allergic rhinitis, rhinoconjunctivitis, asthma, atopic dernatitis, and systhemic anaphylactic reactions.

In embodiments method 100 is applied to treat autoimmune diseases, including: rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis, Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura.

Step 2 of providing a phototherapeutical apparatus includes providing a phototherapeutical apparatus with a light source, which is capable of emitting ultraviolet A, ultraviolet B and visible light, and any combination of these lights.

The light source includes in some embodiments a quartz bulb with electrodes, the electrodes defining a discharge volume of 0.1 mm³ to 3 mm³. The light generated in such a small discharge volume can be coupled into optical fibers directly and efficiently, without optical lenses or other interfaces. Further details of the phototherapeutical apparatus were described in the two incorporated patents.

Step 4 of determining a minimal erythema dose includes irradiating a group of test areas with different doses; examining the test areas after a predetermined time for a predetermined reaction; and recording the dose corresponding to a test area exhibiting the predetermined reaction. In embodiments, a group of skin test areas is used, which were not exposed to sun recently. The predetermined reaction can be the reddening of the targeted area, also known as erythema, after a predetermined time. For example, in some embodiments the irraditated target areas are investigated 24 hours after the irradiation for reddening and the dose corresponding to the reddened area is recorded. In some cases the irradiated target areas were investigated after about 6 to 48 hours.

Step 4 can further include calculating the minimal ertyhema dose from the recorded dose by applying a correction factor corresponding to the target surface. This correction factor can be within the range of about 0.1 and about 10, and is based on the specifics of the target areas.

Step 6 of performing phototherapy includes irradiating a target surface of a patient with a dose, determined from the minimal erythema dose. For example, the corrected or the uncorrected minimal erythema doses can be applied depending on the specifics of the treatment and the target area. In some embodiments, the dose of the phototherapy is choosen without determining the minimal erythema dose, with a fixed dose between about 20 mJ/cm² and about 1000 mJ/cm², based on the patient's skin type.

The phototherapy of step 6 increases the IL-10 production in the target tissue and influences the functions of dendritic cells. This may result in a “tolerogenic” environment, when this target surface will be exposed to an antigen.

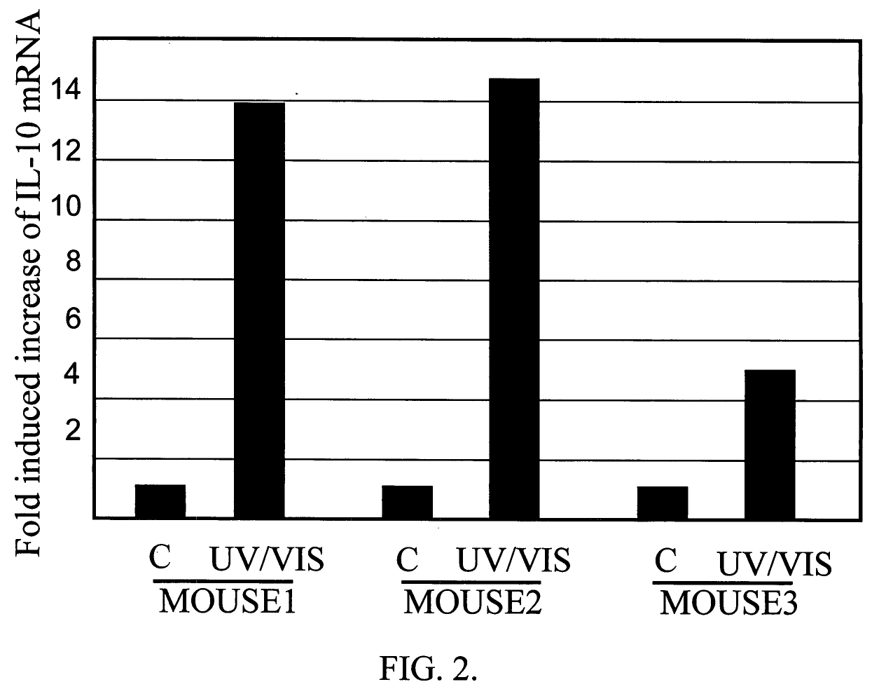

FIG. 2 illustrates the results in a particular embodiment. In the corresponding experiments the oral mucosa of groups of mice were irradiated with UVB light in dosages of 0.1× MED (minimum ertyhema dosage) to 6× MED, and the tissue IL-10 level was measured in different time intervals. In other experiments UVA or visible light was used, applied by the phototherapeutical apparatus of step 2. It was found that UVB light induced a dose and time-dependent increase in the IL-10 production in the oral mucosa of the mice, compared to the control mice. For example, IL-10 mRNA production was increased 4-14 fold by ultraviolet and visible light irradiation (UV/VIS), compared to control mice, indicated by C. IL-10 is known to modulate the antigen-induced immune response in the direction of tolerance. Therefore, using UVB light improves the efficiency of immunotherapy. If an antigen gets into contact with a tissue environment, where IL-10 is present in a higher level, the antigen might induce tolerance instead of inducing allergy. Another set of experiments showed that not only UVB, but UVA, or combination of UVB with UVA and visible light was also effective in inducing IL-10 production in the mucosa, suggesting, that other wavelenghts might also be effective in inducing tolerance.

A tipical dose from UVB or combined UVB+UVA light that resulted in increased IL-10 production varied between about 10 mJ/cm² to about 1000 mJ/cm². Depending on the dose and wavelength of the UV light, the peak IL-10 production in the mucosa was observed between 24 to 72 hours after irradiation.

Step 8 of performing immunotherapy includes exposing the previously irradiated target surface to at least one of a naturally occurring allergen or by exposing the previously irradiated target surface by administering at least one of an allergen or any type of an antigen that is used for conventional immunotherapy. In some embodients Step 8 includes exposing the irradiated surface to foreign (not self) antigens.

It is an aspect of embodiments of the invention that the combination of step 6 of performing a phototherapy with step 8 of performing an immunotherapy increases the efficacy of method 100. Step 6 of the method can increase the level of interleukin-10 and in this environment Step 8 of the method can induce higher number of T regulatory cells or can increase the production of blocking immunoglobulin G type antibodies, among others.

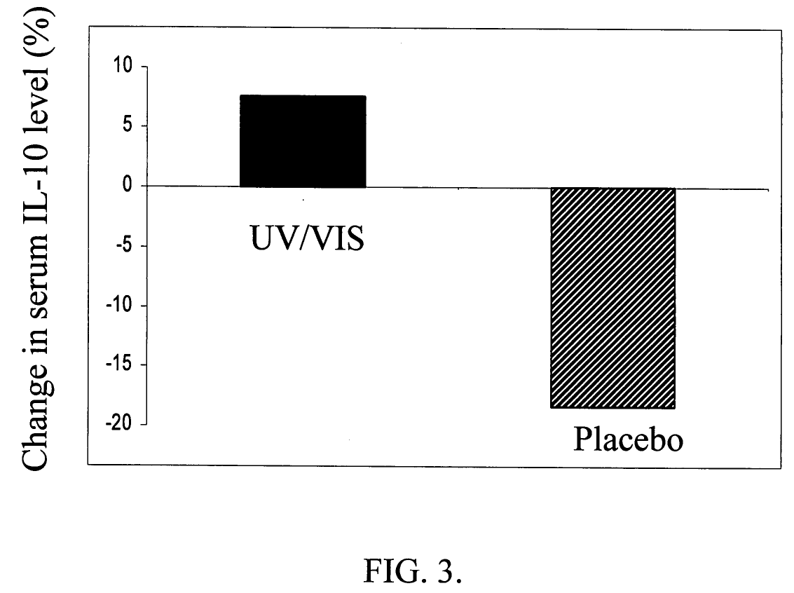

FIG. 3 illustrates the IL-10 levels related to the phototherapy of step 6. The incorporated patent applications demonstrated that intranasal phototherapy results in an improvement of the clinical symptoms of patients with hay fever. We now found that treatment of allergic rhinitis during pollen season with the ultraviolet and visible light induced a slight increase in the IL-10 serum level and increased the number of IL-10 producing CD4 positive T cells (Tr1 type regulatory T cells, or Treg cells). FIG. 3 illustrates that in some cases the increase of the IL-10 serum level was 5-10%, whereas in the control “placebo” experiments, where low intensity visible light was used, there was a 15-20% decrease when intranasal phototherapy was performed during pollen season. These results suggest that intranasal phototherapy might induce allergen dependent tolerance development during natural allergen exposure. It is already known that Tr1 cells play a role in modulating allergic reactions and are characterized by surface CD4 expression and increased IL-10 production. See D. T. Umetsu, O. Akbari, and R. H. DeKruyff: Regulatory T cells control the development of allergic disease and asthma, J. Allergy Clin. Immunol. vol. 112, pp. 480-487 (2003).

Based on the finding that UV light results in IL-10 production in the mucosa, it was investigated whether intranasal phototherapy might result in a tolerance development to this allergen during pollen season—even in symptom-free patients.

Patients with house dust mite allergy (confirmed with skin prick test) were treated with intranasal mixed ultraviolet/visible light in a dosage of 1.0×MED, three times weekly for 3 weeks. The MED was determined on the patient's non-sun-exposed gluteal area. After achieving the symptom-free state, the intranasal phototherapy was continued once weekly for 4 months with the same dose of 1600 mJ/cm² and the patients were informed not to change their regular environment during this time, in order to keep their natural allergen exposure on the same level. The goal of this procedure was to induce tolerance against the house dust mite. After this photoadjuvant treatment, the skin prick test reaction was performed again on the patients who showed a significant decrease in the majority of the nasal symptoms. It was found that the allergen induced wheal development was significantly suppressed, suggesting the development of allergen specific tolerance. This suggests that UV irradiation of the symptom-free nasal mucosa, exposed to a natural allergen, might result in the development of allergen specific tolerance.

A difference of embodiments of the presently described photoadjuvant method and the method to treat allergic rhinitis described in the incorporated patents is that in embodiments of the present invention an aim of the treatment of the clinically noninvolved mucosa was not to prevent a disease, as it was in the incorporated patents, but to induce tolerance, i.e. to cure the disease. Another difference is that in some of the preventive methods of the incorporated patents the patients are kept in an allergen-free environment, whereas embodiments of the present method 100 work efficiently when the patients are exposed to allergen.

Step 8 of performing immunotherapy in some embodiments includes using allergens, which are used for conventional immunotherapies to induce allergen specific tolerance. The photoadjuvant immunotherapy with natural allergen exposure has the benefit that there is no need to provide often expensive (isolated, recombinant or modified) allergens and works also against multiple allergies and in cases where artificial allergens do not exist. However, in this embodiment of method 100 the natural allergen levels show great variability and constant stimulation of the immunological tolerance is hardly possible. To overcome this problem, embodiments of method 100 achieve allergen dependent tolerance by combining the phototherapy with administering conventional allergens that are used in immunotherapy.

In some tests, a target tissue was irradiated with ultraviolet light (nasal mucosa, oral mucosa or skin) 1-3 days before performing the conventional immunotherapy. The UV dose depended on the patients MED level, determined before starting the tolerance induction. Depending on the dose and wavelenghts of the UV light, the immunotherapy was performed just after the UV irradiation, or several days thereafter.

This embodiment of method 100 might be used not only to increase the efficacy of conventional immunotherapies for treating allergic diseases, but also to increase the efficacy of tolerance induction with a specific autoallergen to treat autoimmune diseases in general.

In some embodiments of method 100, recurrent spontaneous abortion (RSA) is treated by inducing the tolerance against paternal antigens in a woman, who intends to be pregnant from an individual. In this embodiment, step 8 includes isolation of paternal lymphocytes from the blood of the individual and injecting the paternal lymphocytes intradermally into the person who wants to be pregnant from the given individual. The number of injected lymphocytes might be in the range of about 100.000 cells to about 1 million cells. In some embodiments step 8 is practiced in the same time on four different sites that have been irradiated previously in step 6. For the treatment of RSA step 6 might include irradiation of the skin with 2-4 times the minimal erythema dosis and step 8 is practiced 24-96 hours after irradiation.

For the treatment of RSA, method 100 is repeated sometimes in four weekly intervals up to usually a maximum of 6-8 times, when tolerance to paternal antigens is usually achieved. After this period, the woman is advised to get pregnant. In some embodiments in step 8 instead of lymphocytes, other types of cells, such as sperm cells are used, and step 6 will be practiced on the vaginal or rectal or oral mucosa. In this embodiment step 8 is performed by administration of the sperm cells to the surface of the vaginal or rectal mucosa.

Method 100 might also be used to develop tolerance in a recipient individual, who needs organ or cell transplantation, against antigens derived from a different donor individual, to prevent or suppress rejection reactions after organ or cell transplantation. In this embodiment, lymphocytes or other types of cells from the donor are used in step 8 to develop tolerance against donor antigens in the recipient. In some embodiments, method 100 will be repeated one to four weekly intervals before performing organ or cell transplantation to achive tolerance against donor antigens. Step 8 can include exposing the irradiated target surface by administering an antigen by an intradermal, subcutaneous route, or administration of an antigen to the surface of the nasal, oral, vaginal, or rectal mucosa.

In some embodiments of method 100, step 6 of performing phototherapy includes irradiating a target surface of a body cavity, for example nasal cavity, mouth cavity, rectum, vaginal mucosa, portio, uterus, and conjunctiva.

In embodiments, the nasal mucosa is irradiated within the nasal cavity. These embodiments offer a treatment of common allergic diseases, including allergic rhinitis (hay fever), allergic rhinoconjunctivitis, allergic asthma, atopic dermatitis, and systhemic anaphylaxis.

In embodiments, where the nasal mucosa is irradated, a correction factor of about 0.1 to 10 may be applied to the MED. Correspondingly, the applied dose of the irradiation can be in the range of about 10 mJ/cm² to about 1000 mJ/cm². The nasal mucosa may be irradiated with a combination of ultraviolet A, ultraviolet B and visible light.

In embodiments, step 8 of performing phototherapy on the nasal mucosa includes enhancing the level of interleukin-10 in the nasal mucosa by irradiation.

In the above embodiments the steps of the method can be performed repeatedly to reach a desired therapeutical result. Also, the steps can be performed in different order.

Method 100 is also suitable as a prevention protocol, applied to patients who do not display the symptoms yet.

In the following two examples of method 100 are described in some detail.

### EXAMPLE 1

A patient had severe perennial allergic rhinitis because of house dust mite allergy. As an application of method 100, the minimal erythema dose measured on the patient's gluteal area with a light source that emitted mixed UV and visible light containing 5% UVB, 25% UVA and 70% visible light. Subsequently, intranasal phototherapy was started. Both nasal cavities were illuminated with a total dose of 16 J once a week. Irradiation of the left and right nasal cavities lasted for 5 minutes. Three weeks after starting the therapy, the patient was free of symptoms. However, intranasal phototherapy was continued for an additional eight weeks with the same dose of mixed UV and visible light. The patient was asked not to change his environment throughout the study, in order to be exposed continuously to the same allergen. After stopping the therapy, the patient remained symptom-free for six month. The skin prick test reaction was measured before the phototherapy and twelve weeks after starting the phototherapy, and there was a significant decrease in the house dust mite-induced wheat formation in the skin prick test reaction, suggesting the development of tolerance.

### EXAMPLE 2

A patient having ragweed allergy was treated with photoadjuvant immunotherapeutical method 100. The minimal erythema dose (MED) was measured on the patient's glutal area using a 308 nm xenon hloride excimer laser and was found to be 200 mJ/cm². The sublingual area of the patient was then irradiated (sublingual phototherapy) with the 308 nm excimer laser in a dose of 1×MED (200 mJ/cm²). One day after irradiation, sublingual allergen specific immunotherapy was started with Pangramin oral as suggested by the manufacturer. The sublingual phototherapy was continued once a week for one year. The irradiation dose was gradually increased, monthly by 25%. The sublingual immunotherapy was continued for one year as suggested by the manufacturer. After one year, in the next ragweed season, the patient had less symptoms of allergic rhinitis and also the ragweed induced wheal formation in the skin prick test reaction decreased significantly.

It is recognized that the invention has been described in relation to specific embodiments. However, several variations will be obvious to a person skilled in the art. These variations and combinations of equivalents are understood to be within the scope of the invention.

What is claimed is:

1. A photoadjuvant immunotherapeutical method, the method comprising the steps of:
providing a phototherapeutical apparatus, comprising a light source, an optical guidance system, and a patient interface;
determining a minimal erythema dose;
performing phototherapy by irradiating a target surface of a body with the phototherapeutical apparatus; and
performing immunotherapy by exposing the irradiated target surface to an antigen.

2. The method of claim 1, wherein the antigen is one of an allergen, a modified allergen, a synthetic allergen, an autoantigen, a foreign antigen, a donor antigen, a paternal antigen and a modified antigen.

3. The method of claim 1, wherein the method is applied to treat allergic diseases, comprising at least one of:
allergic rhinitis, rhinoconjunctivitis, asthma, atopic dermatitis, and systhemic anaphylactic reactions.

4. The method of claim 1, wherein the method is applied to treat autoimmune diseases, comprising at least one of:
rheumatoid arthritis, multiple sclerosis, juvenile-onset diabetes, systemic lupus erythematosus, autoimmune uveoretinitis, autoimmune vasculitis, bullous pemphigus, myasthenia gravis, autoimmune thyroiditis, Hashimoto's disease, Sjogren's syndrome, granulomatous orchitis, autoimmune oophoritis, Crohn's disease, sarcoidosis, rheumatic carditis, ankylosing spondylitis, Grave's disease, and autoimmune thrombocytopenic purpura.

5. The method of claim 1, the step of providing a phototherapeutical apparatus comprising
providing the light source with capability of emitting at least one of an ultraviolet A, ultraviolet B and visible light.

6. The method of claim 1, the step of providing a phototherapeutical apparatus comprising
providing light source, comprising a quartz bulb with electrodes, the electrodes defining a discharge volume in the range of about 0.1 mm³ to about 3 mm³.

7. The method of claim 1, the step of determining a minimal erythema dose comprising:
irradiating a plurality of test areas with different doses;
examining the test areas after a predetermined time for a predetermined reaction; and
recording the dose corresponding to a test area exhibiting the predetermined reaction.

8. The method of claim 7, the step of determining a minimal erythema dose comprising
calculating the minimal erythema dose from the recorded dose by applying a correction factor corresponding to the target surface.

9. The method of claim 1, the step of performing phototherapy comprising
irradiating a target surface of a patient with a dose, determined from the minimal erythema dose.

10. The method of claim 1, the step of performing phototherapy comprising
increasing a tolerance of a patient's body toward an antigen.

11. The method of claim 10, the step of increasing the tolerance comprising at least one of:
increasing the number of T regulatory cells in the patient's body;
increasing the level of interleukin-10 in at least one of the target tissue and the patient's body; and
increasing the number of blocking immunoglobulin G type antibodies in the patient's body.

12. The method of claim 1, the step of performing immunotherapy comprising at least one of:
exposing the irradiated target surface to a naturally present antigen; and
exposing the irradiated target surface to an administered antigen.

13. The method of claim 12, the step of exposing the irradiated target surface to an administered antigen comprising:
administering an antigen by at least one of an intradernal route, subcutaneous route, intranasal route, and sublingual route.

14. The method of claim 1, the step of performing phototherapy comprising
irradiating a target surface of a body cavity.

15. The method of claim 14, wherein the body cavity is one of:
a nasal cavity, a mouth cavity, a rectum, a vaginal mucosa, a portio, a uterus, and a conjunctiva.

16. The method of claim 1, the step of performing phototherapy comprising
irradiating a target surface in a nasal mucosa.

17. The method of claim 16, the step of determining a minimal erythema dose comprising:
irradiating a plurality of test areas with different doses;
examining the test areas after a predetermined time for erythema; and
recording the dose corresponding to a test area exhibiting erythema.

18. The method of claim 17, wherein the predetermined time is between about 6 hours and about 48 hours.

19. The method of claim 17, the step of determining a minimal ertyhema dose comprising
calculating the minimal erythema dose from the recorded dose by applying a correction factor between 0.1 and 10 to the recorded dose.

20. The method of claim 16, the step of performing phototherapy comprising
irradiating the target surface with dose in the range of about 10 mJ/cm² to about 1000 mJ/cm².

21. The method of claim 16, the step of performing phototherapy comprising
irradiating the target surface with a combination of ultraviolet A, ultraviolet B and visible light.

22. The method of claim 16, the step of performing phototherapy comprising
enhancing a level of interleukin-10 in the nasal mucosa by irradiating the target surface in the nasal mucosa.

23. The method of claim 1, the method further comprising
repeating the steps of the photoadjuvant immunotherapeutical method to reach a predetermined therapeutical result.

24. The method of claim 1, the method further comprising
applying the method to a patient not displaying symptoms.

25. The method of claim 1, wherein the method is applied to reduce recurrent spontaneous abortion.

26. The method of claim 25, wherein the step of performing immunotherapy comprises:
isolating paternal lymphocytes from the blood of an individual; and
injecting the paternal lymphocytes intradermally into a person.

27. The method of claim 26, wherein
the step of performing phototherapy comprises irradiating a skin area with about 2 to 4 times the minimal erythema dose; and
the step of performing immunotherapy comprises injecting a number of lymphocyte cells in a range of about 100,000 cells to about 1 million cells in a time range of about 24 to 96 hours after the irradiation.

28. The method of claim 25, wherein the step of performing immunotherapy comprises:
isolating paternal sperm cells from an individual; and
administering the paternal sperm cells on the vaginal or rectal or oral mucosa of a person.

29. The method of claim 1, wherein the method is applied to prevent a rejection reaction in one of an organ and a cell transplantation.

30. The method of claim 29, wherein the step of performing immunotherapy comprises:
administering an antigen by an intradermal subcutaneous route; and
administering an antigen to a surface of one of a nasal, oral, vaginal and rectal mucosa.
